(19) United States
(12) Patent Application Publication
(10) Pub. No.: US 2014/0277059 A1
(43) Pub. Date: Sep. 18, 2014

(54) APPARATUS FOR PUNCTURING BALLOON IN AIRWAY DILATION SHAFT

(71) Applicant: Acclarent, Inc., (US)

(72) Inventors: Sivette Lam, Milpitas, CA (US); Lawrence D. Wasicek, San Jose, CA (US); Ketan P. Muni, San Jose, CA (US); Show-Mean Wu, Fremont, CA (US); Andy Nguyen, San Jose, CA (US); Nicholas E. Sherman, Waxhaw, NC (US); Randy S. Chan, San Jose, CA (US)

(21) Appl. No.: 13/795,857

(22) Filed: Mar. 12, 2013

(51) Int. Cl. A61M 29/02

(52) U.S. Cl. CPC A61M 29/02
USPC 606/192

(57) ABSTRACT

A dilation system includes a balloon catheter and a puncturing assembly. The balloon catheter includes a shaft, a dilator coupled to a distal end of the shaft, and a lumen extending through the shaft and the dilator. The dilator is expandable from a collapsed state to an expanded state. The puncturing assembly includes at least one cutting member. The puncturing assembly is positionable within the lumen of the balloon catheter or along an exterior of the shaft when the puncturing assembly is in a first position. The puncturing assembly transitions to a second position to puncture the lumen of the balloon catheter and/or to puncture the dilator, to thereby drain fluid from the dilator.

### BACKGROUND

In some instances, it may be desirable to dilate an anatomical passageway in a patient. This may include dilation of ostia of paranasal sinuses, dilation of a patient's airway (e.g., to treat a stenosis within the larynx), dilation of the nasal cavity, dilation of the Eustachian tube, dilation of other passageways within the ear, nose, or throat, dilation of blood vessels, dilation of the urethra, etc. One method of dilating anatomical passageways includes using a guide wire and catheter to position an inflatable balloon within the anatomical passageway, then inflating the balloon with a fluid (e.g., saline) to dilate the anatomical passageway.

Airway stenosis (or “airway narrowing”) is a medical condition that occurs when some portion of a patient's airway becomes narrowed or constricted, thus making breathing difficult. A stenosis may occur in any part of the airway including the larynx, trachea, bronchi, or a combination of any of the above mentioned regions. Both adults and children may develop a stenosis. In some instances, a stenosis is caused by intubation, which is when a tube is placed in the airway for ventilation/breathing assistance in a patent who cannot breathe. Intubation for prolonged periods of time may traumatize the airway, causing scar tissue formation that forms the stenosis.

Therapies for treating an airway stenosis range from endoscopic treatments, such as dilation and laser resection, to open procedures, such as laryngotracheal reconstruction. In one technique, a series of rigid dilators of increasing diameter are pushed down the airway, gradually expanding the constriction but also applying shear forces to the airway. Balloon catheters may also be used to perform dilation of an airway or other anatomical passageway. For instance, the expandable balloon may be positioned within a stenosis in an airway (e.g., larynx, trachea, bronchi, etc.) and then be inflated, to thereby dilate the airway and increase airflow. The dilated airway may then allow for improved breathing. An example of a system that may be used to perform such procedures is described in U.S. Pub. No. 2010/0168511, entitled “System and Method for Dilating an Airway Stenosis,” published Jul. 1, 2010, the disclosure of which is incorporated by reference herein.

While several airway dilation systems have been made and used, it is believed that no one prior to the inventor(s) has made or used the invention described in the appended claims.

### BRIEF DESCRIPTION OF THE DRAWINGS

While the specification concludes with claims which particularly point out and distinctly claim the invention, it is believed the present invention will be better understood from the following description of certain examples taken in conjunction with the accompanying drawings, in which like reference numerals identify the same elements and in which:

FIG. 1 depicts a side view of an exemplary system for dilating a stenosis in the airway, including a balloon catheter and a stylet;

FIG. 2 depicts a side view of the stylet of FIG. 1;

FIG. 3A depicts a cross sectional view of another exemplary balloon catheter;

FIG. 3B depicts a magnified view of the balloon catheter of FIG. 3A, taken along line 3B of FIG. 3A;

FIG. 4A depicts a cross sectional view of the system of FIG. 1 being introduced into an airway in a collapsed state, with the balloon positioned within a stenosis;

FIG. 4B depicts a cross sectional view of the system of FIG. 4A in a dilated state, with the balloon inflated to dilate the stenosis;

FIG. 5 depicts a perspective view of an exemplary balloon catheter puncturing assembly;

FIG. 6A depicts a cross sectional side view of the puncturing assembly of FIG. 5 being introduced into a balloon catheter in a collapsed state;

FIG. 6B depicts a cross sectional side view of the puncturing assembly of FIG. 6A in an expanded state;

FIG. 6C depicts a cross sectional side view of the puncturing assembly of FIG. 6A in a collapsed state, showing the balloon catheter punctured;

FIG. 7A depicts a cross sectional side view of another exemplary puncturing assembly being introduced into a balloon catheter in a collapsed state;

FIG. 7B depicts a cross sectional side view of the puncturing assembly of FIG. 7A in an expanded state;

FIG. 7C depicts a cross sectional side view of the puncturing assembly of FIG. 7A in a collapsed state, showing the balloon catheter punctured;

FIG. 8A depicts a cross sectional side view of another exemplary puncturing assembly being introduced into a balloon catheter in a collapsed state;

FIG. 8B depicts a cross sectional side view of the puncturing assembly of FIG. 8A in an expanded state;

FIG. 8C depicts a cross sectional side view of the puncturing assembly of FIG. 8A in a collapsed state, showing the balloon catheter punctured;

FIG. 9 depicts a cross sectional side view of another exemplary puncturing assembly;

FIG. 10A depicts a cross sectional end view of the puncturing assembly of FIG. 9, introduced into a balloon catheter in an collapsed state;

FIG. 10B depicts a cross sectional end view of the puncturing assembly of FIG. 10A in an expanded state;

FIG. 10C depicts a cross sectional end view of the puncturing assembly of FIG. 10A in a collapsed state, showing the balloon catheter punctured;

FIG. 11A depicts a cross sectional side view of another exemplary puncturing assembly being introduced into a balloon catheter in a collapsed state;

FIG. 11B depicts a cross sectional side view of the puncturing assembly of FIG. 11A in an expanded state;

FIG. 11C depicts a cross sectional side view of the puncturing assembly of FIG. 11A in a collapsed state, showing the balloon catheter punctured;

FIG. 12A depicts an end view of a cutting assembly of the puncturing assembly of FIG. 11A in a collapsed state;

FIG. 12B depicts an end view of the cutting assembly of FIG. 12A in an expanded state;

FIG. 13A depicts a cross sectional side view of another exemplary puncturing assembly being introduced into a balloon catheter in a collapsed state;

FIG. 13B depicts a cross sectional side view of the puncturing assembly of FIG. 13A in an expanded state;

FIG. 13C depicts a cross sectional side view of the puncturing assembly of FIG. 13A in a collapsed state, showing the balloon catheter punctured;

FIG. 14A depicts a cross sectional side view of another exemplary puncturing assembly being introduced into a balloon catheter in a collapsed state;

FIG. 14B depicts a cross sectional side view of the puncturing assembly of FIG. 14A in an actuated state;

FIG. 14C depicts a cross sectional side view of the puncturing assembly of FIG. 14A in a collapsed state, showing the balloon catheter punctured;

FIG. 15A depicts a cross sectional side view of another exemplary balloon deflation assembly being introduced into a balloon catheter in a non-activated state;

FIG. 15B depicts a cross sectional side view of the balloon deflation assembly of FIG. 15A in an activated state, showing an opening burned in the balloon catheter;

FIG. 16A depicts a side elevational view of another exemplary puncturing assembly in a retracted state before actuation;

FIG. 16B depicts a side elevational view of the puncturing assembly of FIG. 16A in an actuated state;

FIG. 16C depicts a side elevational view of the puncturing assembly of FIG. 16A in a retracted state after actuation, showing the balloon punctured;

FIG. 17A depicts a side elevational view of another exemplary puncturing assembly in a retracted state before actuation;

FIG. 17B depicts a side elevational view of the puncturing assembly of FIG. 17A in an actuated state; and

FIG. 17C depicts a side elevational view of the puncturing assembly of FIG. 17A in a retracted state after actuation, showing the balloon punctured.

The drawings are not intended to be limiting in any way, and it is contemplated that various embodiments of the invention may be carried out in a variety of other ways, including those not necessarily depicted in the drawings. The accompanying drawings incorporated in and forming a part of the specification illustrate several aspects of the present invention, and together with the description serve to explain the principles of the invention; it being understood, however, that this invention is not limited to the precise arrangements shown.

### DETAILED DESCRIPTION

The following description of certain examples of the invention should not be used to limit the scope of the present invention. Other examples, features, aspects, embodiments, and advantages of the invention will become apparent to those skilled in the art from the following description, which is by way of illustration, one of the best modes contemplated for carrying out the invention. As will be realized, the invention is capable of other different and obvious aspects, all without departing from the invention. Accordingly, the drawings and descriptions should be regarded as illustrative in nature and not restrictive.

It will be appreciated that the terms “proximal” and “distal” are used herein with reference to a clinician gripping a handpiece assembly. Thus, an end effector is distal with respect to the more proximal handpiece assembly. It will be further appreciated that, for convenience and clarity, spatial terms such as “top” and “bottom” also are used herein with respect to the clinician gripping the handpiece assembly. However, surgical instruments are used in many orientations and positions, and these terms are not intended to be limiting and absolute.

It is further understood that any one or more of the teachings, expressions, versions, examples, etc. described herein may be combined with any one or more of the other teachings, expressions, versions, examples, etc. that are described herein. The following-described teachings, expressions, versions, examples, etc. should therefore not be viewed in isolation relative to each other. Various suitable ways in which the teachings herein may be combined will be readily apparent to those of ordinary skill in the art in view of the teachings herein. Such modifications and variations are intended to be included within the scope of the claims.

I. Overview of Exemplary Balloon Dilation Catheter System

FIG. 1 shows an exemplary dilation catheter system (8), which may be used to dilate a stenosis in an airway; or to dilate some other anatomical passageway (e.g., within the ear, nose, throat, cardiovascular system, etc.). At least part of system (8) may be constructed and operable in accordance with at least some of the teachings of U.S. Pub. No. 2010/0168511, the disclosure of which is incorporated by reference herein. It should be understood that dilation catheter system (8) may be used to dilate either a naturally occurring passageway in a patient or a surgically created passageway in a patient.

Dilation catheter system (8) of this example comprises a balloon catheter (10) and a stylet (22). Balloon catheter (10) comprises a shaft (12) positioned between a hub (14) and a balloon (18). Balloon (18) is coupled to a distal end of shaft (12) and is configured to receive fluid through balloon catheter (10). Stylet (22) is slidably positioned through balloon catheter (10). In some versions, at least a portion of stylet (22) has a greater stiffness than at least a portion of balloon catheter (10), such that when stylet (22) is bent and inserted within balloon catheter (10), balloon catheter (10) at least partially conforms to the shape of stylet (22). In a dilation procedure, stylet (22) is used to advance balloon catheter (10) within an airway or targeted anatomical passageway (e.g., at a stenosis site). Balloon (18) may then be actuated to an expanded state to open or dilate the targeted anatomical passageway. Balloon (18) may then be actuated back to a collapsed state such that balloon (18) is deflated. This process may be repeated to dilate several anatomical passageways.

A. Exemplary Stylet

FIG. 2 shows stylet (22) in greater detail. Stylet (22) comprises a core member (26) with a proximal section (28) and a distal section (30). A coil (32) is disposed around at least part of distal section (30) of core member (26). A luer lock member (35) is coupled with a proximal end of core member (26) for coupling with a hub (36) on balloon catheter (10). In some versions, stylet (22) does not include a coil (32). Core member (26) and/or coil (32) may be formed of nitinol, stainless steel, or other biocompatible materials. Distal portion (30) of stylet (22) includes a bend or curve (34) that is stiff enough to bend balloon catheter (10) during the placement of balloon catheter (10) within the airway of the patient. In some versions, stylet (22) may be provided in a generally straight configuration. Stylet (22) may be pre-formed to have a bend (34), or stylet (22) may be malleable, such that a user may bend stylet (22) and stylet (22) maintains the user-created bend. This malleability allows a user to adjust a bend angle according to the airway anatomy of a particular patient. Proximal section (28) of stylet (22) may be generally stiff, a distal section (30) may be generally malleable, and an extreme distal portion may be atraumatic and very flexible or even floppy. This variation in flexibility along the length of stylet (22) may be achieved by using different materials, such as stainless steel and nitinol. Alternatively, one material, such as stainless steel, may be used and the diameter of stylet (22) may be altered to achieve the variation in flexibility along the length of stylet (22).

Stylet (22) has an overall length approximately as long or slightly longer than balloon catheter (10). In some versions, stylet (22) includes an atraumatic, flexible distal tip portion that extends distally out of balloon catheter (10) when stylet (22) is fully disposed within catheter (10). By way of example only, this tip portion extend distally out of catheter (10) by about 0.25 cm to about 8 cm, or more particularly by about 1 cm to about 5 cm, and may facilitate the ability of a user to advance system (8) through a patient's airway atraumatically. The overall length of stylet (22) may vary from about 30 cm to about 80 cm, such as from about 45 cm to about 60 cm. Of the overall length, a flexible distal portion of stylet (22) may be from about 5-20 cm, such as from about 10-15 cm. Bend (34) may have any suitable angle, such as from greater than 0 degrees to about 20 degrees. The diameter of stylet (22) may be less than about 1.3 mm, such as 0.9 mm or less. The diameter may decrease distally to about 0.13 mm+/−0.013 mm. Of course, the foregoing dimensions are mere examples. Any other suitable dimensions may be used.

Stylet (22) may be attached to balloon catheter (10), or stylet (22) may be removably connected to balloon catheter (10). Stylet (22) comprises a luer lock member (35) with threads on proximal section (28) that screw into opposing threads disposed on a luer (36) of balloon catheter (10). In some versions, balloon catheter (10) may include a locking mechanism (not shown) to lock stylet (22) in position within catheter (10). The locking mechanism can be any mechanical device, including a lever, a ball and pin, a luer, etc. All or part of distal section (30) of stylet (22) may extend out of the distal end of catheter (10). Stylet (22) may be locked to balloon catheter (10) at different positions or lengths so the distal end of stylet (22) extends out of or is positioned within balloon catheter (10) at different lengths. The length, diameter(s) and stiffness characteristics of stylet (22) may be varied in different embodiments to confer different performance characteristics to the overall system (8).

Use of stylet (22) to insert balloon catheter (10) helps to guide the distal end of balloon catheter (10) through the airway of the patient and to the stenotic region. Stylet (22) provides increased steerability during advancement of balloon catheter (10). Torquability of balloon catheter (10) is also increased when using stylet (22). In some versions, luer lock member (35) of stylet (22) and luer (36) of balloon catheter (10) mate together, so that stylet (22) and balloon catheter (10) may be rotated together and thus steered into a constricted portion of an airway.

In some versions, stylet (22) may have a light emitting portion, such as a light emitting distal end or tip. For example, stylet (22) may include one or more light fibers to transmit light from a light source attached to the proximal end of stylet (22) to its distal end. Light from a light emitting stylet (22) may be used to help a user visualize a patient's airway from the inside using a scope and/or in some cases from the outside via transillumination through the patient's skin. A light emitting guidewire device that may be used or modified to achieve such an illuminating stylet (22) is the Relieva Luma™ Sinus Illumination Guidewire/System, manufactured by Acclarent, Inc. of Menlo Park, Calif. Such an illuminating stylet (22) may have any of the features described above with the additional feature of light emitting capability.

B. Exemplary Balloon Catheter

FIGS. 3A and 3B show an exemplary balloon catheter (50). Balloon catheter (50) is similar to balloon catheter (10) and may be readily incorporated for use with system (8). Balloon catheter (50) comprises a catheter shaft (52) having an outer shaft member (54) and an inner shaft member (56). An inflatable balloon (58) is attached to shaft (52) at a proximal attachment point (62) and at a distal attachment point (64). A hub (60) is coupled to a proximal end of shaft (52) and comprises a stylet port (66) and an inflation port (68). In the present example, outer shaft member (54) is disposed over a portion of inner shaft member (56), with inner shaft member (56) continuing to the distal end of catheter (50). Balloon (58) is attached at proximal attachment point (62) to outer member (54) and at distal attachment point (64) to inner shaft member (56), either via adhesive or other attachment means. Thus, an inflation lumen (too small to view on FIG. 3A) is formed between inner and outer shaft members (56, 54), with inflation fluid passing into catheter (50) from an inflation device (not shown), through inflation port (68), into the inflation lumen, and into balloon (58). Stylet (22) generally resides within an inner lumen (57) of inner shaft member (56), as shown in FIG. 3B. Balloon (58) may comprise an inner wall coupled with inner shaft member (56), or balloon (58) may comprise only an outer wall coupled at attachment points (62, 64).

Balloon catheter (50) may have any number of suitable sizes, shapes and configurations. For example, balloon (58) may have different lengths and diameters in different embodiments, to accommodate different patient anatomies. The overall catheter (50) length and diameter may also vary. For example, the overall length of balloon catheter (50) (i.e., from the proximal end of hub (60) to the distal end of catheter shaft (52)) is about 35-70 cm, such as less than or equal to about 50 cm, or about 45 cm+/−5 cm. Catheter (50) may be handled and manipulated with one hand. The working length of balloon (58) in FIGS. 3A and 3B is about 40 mm+/−2 mm. By “working length” it is meant the length between the two tapered portions of balloon (58). In some versions, the working length of balloon (58) may range from between about 10 mm and about 60 mm such as about 16-45 mm. The outer diameter of the fully inflated working length of balloon (58) may also vary. In the present example, balloon (58) has an inflated diameter of about 14.1 mm+/−0.5 mm. In some versions, balloon (58) diameter may range from about 3 mm to about 24 mm, such as about 5-15 mm. A combination of balloon sizes and lengths may be provided, such that a physician may choose an appropriate size for an adult or pediatric patient. In one example, the following combinations may be provided (first dimension is diameter, second is length): 5 mm by 24 mm; 7 mm by 24 mm; 10 mm by 40 mm; and 14 mm by 40 mm. Of course, any of a number of other combinations of sizes of balloons (58) and catheters (50) may be provided.

Any suitable material may be used to form balloon (58). Balloon (58) may be compliant, semi-compliant or non-compliant. Balloon (58) may be made of nylon or other polymer, such as PTFE. In some versions, balloon (58) may include an outer slip-resistant surface, which may be formed by a textured surface or a coating. Such a surface may help prevent slipping of balloon (58) out of an airway structure during inflation and/or may facilitate re-wrapping balloon (58) by hand after deflation if balloon (58) is to be used for a second or subsequent dilation procedure. Examples of such balloons are provided in U.S. Pat. App. No. [FBT DOCKET NO. ACC5059USPSP.600452], entitled “Features to Enhance Grip of Balloon within Airwary,” filed on a date even herewith, the disclosure of which is incorporated by reference herein.

Catheter shaft (52) (outer shaft member (54) and inner shaft member (56)) may be formed of any suitable material. It may be desirable to form shaft (52) from material(s) selected so that shaft (52) is unlikely to kink when bent, such as when bent by stylet (22) and/or a user. One such material, for example, is Pebax, although other polymers may be used. Outer shaft member (54) and/or inner shaft member (56) may also have any suitable color and may include one or more shaft markings. The shaft color and markings may be built into shaft (52) by using a colored material or may be added by applying paint or another colorant. In some versions, shaft (54) may have a dark color, such as black or dark blue, and one or more light colored markings may be applied over the dark shaft (54). In some versions, the markings (not shown) may include direct visualization markings (viewed directly with the naked eye or an endoscope) and/or radiographic markings (viewed with a radiographic device such as intraoperative fluoroscopy). For example, two radiographic markings may be positioned in inner shaft member (56) at the locations of the two working ends of balloon (58), and two direct visualization markings may be positioned on outer shaft (54) approximately 1 cm and 2 cm proximal to proximal attachment point (62). The direct visualization markings may be viewed with a bronchoscope or other endoscope to help a physician approximate the location of balloon (58) relative to anatomy, while the radiographic markings may be viewed with a fluoroscopy device to see where the working ends of balloon (58) are located relative to an airway constriction. Any suitable combination, size and color of markings may be used. One example of shaft color and shaft markings, which could be used or modified for balloon catheter (50), is the Relieva Solo Pro Sinus Balloon Catheter, manufactured by Acclarent, Inc. of Menlo Park, Calif.

Referring again to FIGS. 3A and 3B, inner shaft member (56) extends distally beyond the distal end of balloon (58) by about 1 mm to about 10 mm, such as about 5 mm.+−0.1 mm. This distal end of inner shaft member (56) may act as an atraumatic tip, along with a protruding distal end of stylet (22), which may extend distally further than inner shaft member (56). When a larger diameter balloon (58) is used (10 mm or more, for example), a small segment of inner shaft member (56) toward its distal end may have a larger outer diameter, so that the larger diameter balloon (58) may be adequately bonded to inner shaft member (56) at distal attachment point (64). This keeps inner shaft member (56) small along the rest of its length, while still allowing the larger balloon (58) to be bonded to inner shaft member (56). The larger outer diameter may be formed by adding material to inner shaft member (56) at distal attachment point (64) before bonding. Alternatively, bump tubing may be used, with inner shaft member (56) constructed with the larger diameter built-in at distal attachment point (64).

C. Exemplary Method of Use of the System

FIGS. 4A and 4B show a method for dilating a stenotic region (4) in an airway (2), such as in a case of subglottic stenosis. Dilation system (8) is introduced through the mouth and into the airway of the patient. Optionally, a bronchoscope (not shown) or other scope device may be used to visualize the positioning of dilation system (8). Dilation system (8) may be bent either by the user or by the manufacturer of system (8). For example, stylet (22) may be bent and then inserted into balloon catheter (10, 50), while in other cases stylet (22) and balloon catheter (10, 50) may be bent together, with stylet (22) already residing in catheter (10, 50). The support of stylet (22) and the bend in the overall system (8) may help a physician navigate system (8) through the patient's airway to position balloon (18, 58) within at least a portion of stenotic region (4). As shown in FIG. 4A, inflatable balloon (18, 58) of the catheter (10, 50) is in an unexpanded configuration during advancement and placement of balloon catheter (10, 50). As shown in FIG. 4B, once balloon (18, 58) is positioned within stenotic region (4) of the airway (2), inflatable balloon (18, 58) is inflated to dilate stenotic region (4). Balloon (18, 58) is then deflated to enable removal from airway (2). By way of example only, balloon (18, 58) may be deflated by actively drawing the fluid from balloon (18, 58); by venting the fluid in balloon (18, 58), allowing the inward pressure imposed by airway (2) to drive fluid from balloon (18, 58); or in any other suitable fashion as will be apparent to those of ordinary skill in the art in view of the teachings herein.

In some versions, stylet (22) remains in balloon catheter (10, 50) during inflation of balloon (18, 58). Maintaining stylet (22) in catheter (10, 50) during inflation may give catheter (10, 50) added column strength and help maintain the position of balloon (18, 58) within stenotic region (4), thus avoiding slipping. In some versions, stylet (22) is removed from balloon catheter (10, 50) before inflating. Stylet (22) may be removed from balloon catheter (10, 50) after balloon catheter (10, 50) is properly positioned within airway (2) of the patient, or stylet (22) can be removed after stenosis (4) has been dilated but before removing balloon catheter (10, 50) from the patient.

Inflatable balloon (18, 58) may be inflated more than once to dilate stenotic region (4) of airway (2). The physician inflates inflatable balloon (18, 58) to a desired pressure during each dilation of stenosis (4). Proper dilation of stenotic region (4) can be confirmed by visualizing the region with the bronchoscope/endoscope.

II. Exemplary Internal Puncturing Assembly

During some procedures, dilation system (8) may be misused to cause balloon (18, 58) to either deflate slower than desired or remain partially inflated. This may cause balloon (18, 58) to be difficult to remove from the airway; or prevent balloon (18, 58) from being removed from the airway. Accordingly, it may be desirable to provide a puncturing assembly to puncture balloon catheter (10, 50) and/or balloon (18, 58) to help reduce the fluid pressure in balloon (18, 58) quickly, to thereby facilitate removal of balloon (18, 58) from the patient's airway. The puncturing assembly may be inserted within lumen (57) of inner shaft member (56), as shown in FIG. 3B. The outer diameter of the puncturing assembly is sized smaller than the inner diameter of lumen (57). The puncturing assembly may then expand to a diameter larger than the outer diameter of lumen (57) or otherwise breach the wall of lumen (57). The puncturing assembly may thus puncture the wall of lumen (57) to allow fluid to drain proximally or distally through lumen (57). By way of example only, the puncturing assembly may expand more than about 0.038 inches. The fluid may be squeezed through lumen (57) by forces exerted on the exterior of balloon (18, 58) from the anatomical lumen wall. Alternatively, suction may be provided to draw the fluid from lumen (57). In some versions, balloon (18, 58) may have an inner wall that is attached to the exterior inner shaft member (56). If balloon (18, 58) comprises an inner wall, the puncturing assembly may puncture the wall of lumen (57) and the inner wall of balloon (18, 58) to drain the fluid proximally or distally through lumen (57).

In addition to or as an alternative to puncturing the wall of lumen (57), a puncturing assembly may be configured to puncture the outer wall of balloon (18, 58) and allow the fluid to leak into the airway. In instances where the outer wall of balloon (18, 58) is punctured, this may be done from within lumen (57) or from outside of lumen (57). As one merely illustrative example, a puncturing instrument may be inserted through the trachea external to but alongside inner shaft member (56). As another merely illustrative example, a puncturing instrument may be inserted through the sidewall of the trachea (e.g., along a path that is substantially transverse to the trachea) to puncture the outer wall of balloon (18, 58). Regardless of whether balloon (18, 58) is punctured from within lumen (57) or from outside of lumen (57), in some versions, the distal portion of balloon (18, 58) is the area that is punctured. For instance, the puncture site in balloon (18, 58) may be closer to distal attachment point (64) than proximal attachment point (62). Of course, balloon (18, 58) may be punctured at any suitable site in addition to or as an alternative to being punctured in a distal portion of balloon (18, 58).

In some versions, dilation system (8) may comprise a sensing feature to detect strain in balloon catheter (10, 50) and/or elsewhere within dilation system (8). The puncturing assembly may be configured to respond to such a sensing feature to automatically puncture a balloon catheter (10, 50) and/or balloon (18, 58). An example of such a sensing feature that may be incorporated into a dilation system (8) is provided in U.S. Pat. App. No. [FBT DOCKET NO. ACC5057USPSP.0600450], entitled “Apparatus for Sensing and Responding to Strain in Airway Dilation Shaft,” filed on a date even herewith, the disclosure of which is incorporated by reference herein. It should be understood that any of the puncturing features described herein may be incorporated into a system like the one taught in U.S. Pat. App. No. [FBT DOCKET NO. ACC5057USPSP.0600450], such that any of the puncturing features described herein may be automatically actuated in response to a strain sensor or other sensing feature detecting a strain related parameter value exceeding a threshold value. Various suitable ways in which the teachings herein may be combined with the teachings in U.S. Pat. App. No. [FBT DOCKET NO. ACC5057USPSP.0600450] will be apparent to those of ordinary skill in the art.

The following puncturing features may be integrated into a variation of stylet (22). Alternatively, they can be inserted in balloon catheter (10, 50) after stylet (22) is withdrawn. In other words, some versions of puncturing features may also serve a role as stylet (22), while other versions of puncturing features are inserted into lumen (57) after stylet (22) is withdrawn. Still other versions of puncturing features may bear no relation to stylet (22) and may be used while stylet (22) is still disposed in lumen (57). A puncturing assembly may include a tube assembly that is translated to selectively expand the puncturing assembly to puncture a balloon catheter (10, 50). A puncturing assembly may also include puncturing assemblies with translating cutting members to puncture a balloon catheter (10, 50). The examples below provide several versions of puncturing assemblies that may be readily coupled to dilation system (8). In all of the below examples, the wall of lumen (57) and/or balloon (18, 58) may have one or more regions of reduced structural integrity to promote intentional rupture. For instance, such weak regions may include reduced wall thickness, etc.

In some versions, the puncturing assemblies described herein are used in systems where balloon (18, 58) is formed of an elastic/extensible material that is resiliently biased to assume a shrunken, non-inflated configuration, such that the material forming balloon (18, 58) is under increased tension when balloon (18, 58) is in a non-deflated state. In some other versions, the puncturing assemblies described herein are used in systems where balloon (18, 58) is formed of a material that is flexible yet substantially inelastic/non-extensible, such that the material forming balloon does not provide a significant resilient bias. In other words, balloon (18, 58) does not stretch in response to increased fluid pressure inside balloon (18, 58), even though the effective outer diameter of balloon (18, 58) increases in response to increased fluid pressure. Such inelastic versions of balloon (18, 58) may nevertheless be filled with fluid, with the fluid pressure being increased to provide an outwardly directed force via balloon (18, 58), and this process may be referred to as “inflating.” When the pressure of fluid inside balloon (18, 58) is reduced, this process may be referred to as “deflating,” even if the material forming balloon (18, 58) does not elastically shrink, since balloon (18, 58) may nevertheless flexibly collapse in response to reduced fluid pressure. Thus, it should be understood that the use of terms like “inflate,” “inflated,” “deflate,” and “deflated” does not necessarily mean that the material forming balloon (18, 58) undergoes any elastic stretching or shrinking as the fluid pressure within balloon (18, 58) changes.

Various examples of puncturing assemblies will be described in greater detail below, while other suitable puncturing assembly configurations will be apparent to one with ordinary skill in the art in view of the teachings herein. It should also be understood that balloon (18, 58) may be ruptured by intentionally overinflating balloon (18, 58). While the examples described herein are provided mainly in the context of procedures within a patient's airway (e.g., trachea), it should be understood that the teachings herein may be readily applied in various other contexts. By way of example only, the teachings herein may be readily applied in the contexts of naturally occurring or surgically created passageways associated with a patient's ear, nose, throat, or other anatomy. For instance, instruments similar to those described herein may be used within a patient's Eustachian tube, ostia associated with sinus cavities, and/or elsewhere within a patient's anatomy. Other suitable settings of use will be apparent to those of ordinary skill in the art in view of the teachings herein.

A. Exemplary Tapered Mandrel

FIG. 5 shows an exemplary puncturing assembly (100) comprising a tube (120) with cutting members (124) and mandrel (110). Tube (120) has an outer diameter that is sized smaller than inner lumen (57) of inner shaft member (56). Tube (120) is configured to expand to a diameter larger than inner shaft member (56). Tube (120) is configured to collapse back and retain its original diameter. The distal section of tube (120) may be flexible, or the entire length of tube (120) may be flexible. Cutting members (124) are positioned on the exterior of a distal end of tube (120). Although two cutting members (124) are shown, any number of cutting members (124) may be used. Cutting members (124) are configured to pierce through inner shaft member (56). Mandrel (110) is positioned within an opening (122) of tube (120). Mandrel (110) comprises a tapered portion (112) at a distal end of a shaft (114). Tapered portion (112) is configured with a larger diameter at the distal end to a smaller diameter closer to shaft (114). Mandrel (110) is translatable relative to tube (120).

FIGS. 6A-6C show an exemplary use of puncturing assembly (100). As shown in FIG. 6A, puncturing assembly (100) is inserted in a collapsed state within inner shaft member (56) of balloon catheter (10, 50), with cutting members (124) located at a longitudinal position associated with the interior of balloon (18, 58). Puncturing assembly (100) is sized to fit within inner shaft member (56). In the collapsed state, tapered portion (112) of mandrel (110) extends distally from tube (120). As shown in FIG. 6B, mandrel (110) is pulled proximally to expand puncturing assembly (100) to an expanded state. Mandrel (110) is pulled proximally such that tapered portion (112) translates within opening (122) of tube (120) to push the walls of tube (120) outward. This causes cutting members (124) to pierce through inner shaft member (56). In some versions, cutting members (124) further pierce the outer wall of balloon (18, 58). As shown in FIG. 6C, mandrel (110) is pushed distally such that tapered portion (112) again extends distally from tube (120) to allow tube (120) to return to the collapsed state under its own resilient bias. Cutting members (124) disengage from inner shaft member (56). As cutting members (124) disengage from inner shaft member (56), cutting members (124) leave an opening (55) in inner shaft member (56). Fluid may then drain from balloon (18, 58), through opening (55), and through inner shaft member (56). Puncturing assembly (100) may be removed from balloon catheter (10, 50) to more quickly drain balloon (18, 58), or puncturing assembly (100) may remain within balloon catheter (10, 50) while balloon (18, 58) drains.

B. Exemplary Pivoting Cutting Members

FIGS. 7A-7C show another exemplary puncturing assembly (200). Puncturing assembly (200) comprises a tube (220), similar to tube (120) of puncturing assembly (100), shaft (210), and cutting members (224). Shaft (210) is positioned within an opening (222) of tube (220) and is configured to translate within tube (220). Cutting members (224) are positioned at a distal end of shaft (210) and are configured to pivot relative to shaft (210). Cutting members (224) may pivot relative to shaft (210) via pins, and/or cutting members (224) may be resiliently biased outwardly. When the distal end of shaft (210) is positioned within tube (220), tube (220) retains cutting members (224) within shaft (210). When the distal end of shaft (210) is translated past tube (220), cutting members (224) move outwardly from shaft (210). The distal end of shaft (210) may be translated back within tube (220). When the distal end of shaft (210) is translated back within tube (220), cutting members (224) pivot back inwardly into shaft (210). The bottom surface of cutting members (224) may be cammed such that tube (220) engages the cammed surface of cutting members (224) to push cutting members back within shaft (210). Tube (220) is rigid to maintain cutting members (224) within shaft (210). In some versions, cutting members (224) are deployed by retracting tube (220) relative to inner shaft member (56) instead of advancing shaft (210) relative to inner shaft member (56).

As shown in FIG. 7A, puncturing assembly (200) is inserted in a collapsed state within inner shaft member (56) of balloon catheter (10, 50), with cutting members (224) located at a longitudinal position associated with the interior of balloon (18, 58). Puncturing assembly (200) is sized to fit within inner shaft member (56). In the collapsed state, the distal end of shaft (210) is positioned within tube (220) such that cutting members (224) are held within shaft (210) by tube (220). As shown in FIG. 7B, shaft (210) is pushed distally to expand puncturing assembly (200) to an expanded state. The distal end of shaft (210) is pushed to extend from tube (220). As shaft (210) translates distally, cutting members (224) pivot outwardly to extend from shaft (210). This causes cutting members (224) to pierce through inner shaft member (56). In some versions, cutting members (224) further pierce the outer wall of balloon (18, 58). As shown in FIG. 7C, shaft (210) is pulled back proximally such that the distal end of shaft (210) translates within tube (220). As shaft (210) is pulled proximally, tube (220) pushes cutting members (224) to pivot back within shaft (210) to return to a collapsed state. Alternatively, tube (220) may be pushed distally to pivot cutting members (224) back within shaft (210). Cutting members (224) disengage from inner shaft member (56) and leave an opening (55) in inner shaft member (56). Fluid may then drain from balloon (18, 58), through opening (55), and through inner shaft member (56). Puncturing assembly (200) may be removed from balloon catheter (10, 50) to more quickly drain balloon (18, 58), or puncturing assembly (200) may remain within balloon catheter (10, 50) while balloon (18, 58) drains.

C. Exemplary Resilient Cutting Members

FIGS. 8A-8C show another exemplary puncturing assembly (300). Puncturing assembly (300) comprises a tube (320), similar to tube (220) of puncturing assembly (200), and cutting members (326). Cutting members (326) are positioned within an opening (322) of tube (320) and are configured to translate relative to tube (320). Cutting members (326) each comprise a pointed tip (324) at their distal end. Tips (324) are configured to puncture inner shaft member (56). Although two cutting members (326) are shown, any number of cutting members (326) may be used. Cutting members (326) are resiliently biased outwardly such that cutting members (326) extend transversely beyond tube (320) when cutting members (326) are exposed from tube (320). Cutting members (326) may be made of nitinol or any shape memory material. When the distal ends of cutting members (326) are positioned within tube (320), tube (320) retains tips (324) of cutting members (326) within tube (320). When the distal ends of cutting members (326) are translated to extend past tube (320), tips (324) of cutting members (326) travel outwardly beyond tube (320). The distal ends of cutting members (326) may be translated back within tube (320) to again retain tips (324) of cutting members (326) within tube (320). Alternatively, tube (320) may be translated proximally to expose tips (324) of cutting members (324). Tube (320) is rigid to maintain cutting members (326) within tube (320).

As shown in FIG. 8A, puncturing assembly (300) is inserted in a collapsed state within inner shaft member (56) of balloon catheter (10, 50), with cutting members (326) located at a longitudinal position associated with the interior of balloon (18, 58). Puncturing assembly (200) is sized to fit within inner shaft member (56). In the collapsed state, the distal ends of cutting members (326) are positioned within tube (320) such that cutting members (326) are held within tube (320). As shown in FIG. 8B, cutting members (326) are pushed distally to expand puncturing assembly (300) to an expanded state. The distal ends of cutting members (326) are pushed to extend from tube (320). As cutting members (326) translate distally, tips (324) of cutting members (326) travel outwardly to extend beyond tube (320). This causes tips (324) of cutting members (326) to pierce through inner shaft member (56). In some versions, cutting members (326) further pierce the outer wall of balloon (18, 58). As shown in FIG. 8C, cutting members (326) are pulled back proximally such that tips (324) of cutting members (326) translate within tube (320). As cutting members (324) are pulled proximally, tube (320) pushes tips (324) of cutting members (326) back within tube (320) to return to a collapsed state. Cutting members (326) disengage from inner shaft member (56) and leave an opening (55) in inner shaft member (56). Fluid may then drain from balloon (18, 58), through opening (55), and through inner shaft member (56). Puncturing assembly (300) may be removed from balloon catheter (10, 50) to more quickly drain balloon (18, 58), or puncturing assembly (300) may remain within balloon catheter (10, 50) while balloon (18, 58) drains.

D. Exemplary Rotating Cutting Members

FIGS. 9-10C show an exemplary actuatable puncturing assembly (400). Puncturing assembly (400) comprises a tube (420) having a gripping feature (411) at the proximal end of tube (420). A shaft (410) extends through tube (420) such that a portion of shaft (410) is positioned outside the proximal end of tube (420). Shaft (410) comprises an actuator (412) at the proximal end of shaft (410) and a gear (412) at a distal portion of shaft (410) within tube (420). Actuator (412) of the present example comprises a rotation knob. The rotation knob is configured to rotate shaft (410) within tube (420). Gear (412) engages gears (414) of rotation shafts (416). As shaft (410) is rotated, gear (412) causes gears (414) and rotation shafts (416) to rotate. Rotation shafts (416) extend distally within tube (420) through a plate (418). The distal ends of rotation shafts (416) extend through plate (418) to couple with respective cutting members (424). Plate (418) is configured to fix cutting members (424) in a longitudinal position relative to tube (420). As rotation shafts (416) rotate, cutting members (424) rotate. Although two rotation shafts (416) and cutting members (424) are shown, any number of rotation shafts (416) and cutting members (424) may be used.

Puncturing assembly (400) is inserted within inner shaft member (56) in a collapsed state, as shown in FIG. 10A, with cutting members (424) located at a longitudinal position associated with the interior of balloon (18, 58). The collapsed state, cutting members (424) are positioned within the outer perimeter of plate (418). As shown in FIG. 10B, cutting members (424) are rotated to an expanded state. Actuator (412) is rotated to rotate shaft (410) such that gear (412) engages gears (414) to rotate shafts (416). As shafts (416) rotate, cutting members (424) are also rotated. Cutting members (424) rotate to extend through openings (422) of tube (420) to pierce inner shaft member (56). In some versions, cutting members (424) further pierce the outer wall of balloon (18, 58). Actuator (412) may then be rotated in the opposite direction to return cutting members (424) to a collapsed state, as shown in FIG. 10C. Cutting members (424) disengage from inner shaft member (56) and leave an opening (55) in inner shaft member (56). Fluid may then drain from balloon (18, 58), through opening (55), and through inner shaft member (56). Puncturing assembly (400) may be removed from balloon catheter (10, 50) to more quickly drain balloon (18, 58), or puncturing assembly (400) may remain within balloon catheter (10, 50) while balloon (18, 58) drains.

E. Exemplary Translating Cutting Members

FIGS. 11A-12B show another exemplary actuatable puncturing assembly (500). Puncturing assembly (500) comprises a tube (520) similar to tube (420) of puncturing assembly (400). A shaft (510) extends through tube (520) to extend proximally from tube (520). A proximal end of shaft (510) comprises an actuator (512) that may be translated relative to tube (520). For example, a user may grasp gripping feature (511) of tube (520) and actuator (512) to translate actuator (512). The distal end of shaft (510) comprises a first tapered portion (513) that expands to a larger diameter than shaft (510) and a second tapered portion (514) that extends to a distal point. Puncturing assembly (500) further comprises cutting members (524) within tube (510).

Cutting members (524) are positioned adjacent to the point of second tapered portion (514). As shown in FIGS. 12A-12B, cutting members (524) each comprise a cutting edge (527) and a recess (525). Cutting edge (527) is positioned on the outer edge of cutting member (524) adjacent to the wall of tube (520). Recess (525) is positioned on an inner portion of cutting member (524) and is configured to allow tapered portion (514) to translate between recesses (525). Cutting members (524) are coupled with resilient members (528) to bias cutting members (524) inwardly toward each other. As tapered portion (514) translates between cutting members (514), cutting members (524) expand from a collapsed state (FIG. 12A) away from each other to an expanded state (FIG. 12B). Cutting members (524) are thus configured to expand outwardly as shaft (510) translates distally within tube (520). Cutting members (524) are longitudinally maintained within tube (520) by ribs (526) extending into tube (520). Although two cutting members (524) are shown, any number of cutting members (524) may be used.

Puncturing assembly (500) is inserted within inner shaft member (56) in the collapsed state, as shown in FIG. 11A, with cutting members (524) located at a longitudinal position associated with the interior of balloon (18, 58). Tapered portion (514) is proximal to cutting members (524). As shown in FIG. 11B, actuator (512) is translated distally to translate shaft (510) distally. As shaft (510) is translated distally, tapered portion (514) of shaft (510) engages recesses (525) of cutting members (524) to drive cutting members (524) outwardly. As cutting members (524) expand outwardly, cutting members (524) translate through openings of tube (520) to pierce inner shaft member (56). In some versions, cutting members (524) further pierce the outer wall of balloon (18, 58). Actuator (512) is then translated proximally to allow resilient members (528) to return puncturing assembly (500) to a collapsed position, as shown in FIG. 11C. A spring may be used to bias actuator (512) proximally, such that a user only needs to release actuator (512) to return actuator (512) to the proximal position. If tapered portion (513) is translated beyond cutting members (524), tapered portion (513) is tapered such that tapered portion (513) may slide past cutting members (524). Tapered portion (514) is returned proximal to cutting members (524) such that cutting members (524) translate inwardly under the influence of resilient members (528). Cutting members (524) disengage from inner shaft member (56) to leave openings (55). Fluid may then drain from balloon (18, 58), through opening (55), and through inner shaft member (56). Puncturing assembly (500) may be removed from balloon catheter (10, 50) to more quickly drain balloon (18, 58), or puncturing assembly (500) may remain within balloon catheter (10, 50) while balloon (18, 58) drains.

F. Exemplary Retractable Blade

FIGS. 13A-13C show another exemplary actuatable puncturing assembly (600) with a retractable blade (624). Puncturing assembly (600) comprises a tube (620), shaft (610), and actuator (612) similar to tube (520), shaft (510), and actuator (510) of puncturing assembly (500). However, the distal end of shaft (610) is pivotally coupled to retractable blade (624) via pin (626). Blade (624) comprises a tip (628) that is configured to pierce through inner shaft member (56). Blade (624) is also pivotally coupled to tube (620) via pin (627) such that blade (624) is configured to rotate through opening (622) of tube (620) when shaft (610) is translated distally. Actuator (612) is biased proximally by resilient member (614). Resilient member (614) thus biases blade (624) to the retracted position shown in FIG. 13A.

Puncturing assembly (600) is inserted within inner shaft member (56) in the collapsed state, as shown in FIG. 13A, with blade (624) located at a longitudinal position associated with the interior of balloon (18, 58). Actuator (612) is in the proximal position such that blade (624) is positioned within tube (620). As shown in FIG. 13B, actuator (612) is translated distally to translate shaft (610) distally. As shaft (610) is translated distally, blade (624) is pivoted through opening (622) of tube (620). As blade (624) pivots outwardly, blade (624) pierces inner shaft member (56). In some versions, blade (624) further pierces the outer wall of balloon (18, 58). Actuator (612) is then translated proximally to return puncturing assembly (600) to a collapsed position, as shown in FIG. 13C. Shaft (610) translates proximally to pivot blade (624) inwardly back within tube (620). Blade (624) disengages from inner shaft member (56) to leave opening (55). Fluid may then drain from balloon (18, 58), through opening (55), and through inner shaft member (56). Puncturing assembly (600) may be removed from balloon catheter (10, 50) to more quickly drain balloon (18, 58), or puncturing assembly (600) may remain within balloon catheter (10, 50) while balloon (18, 58) drains.

G. Exemplary Deflected Needle

FIGS. 14A-14C show another exemplary puncturing assembly (700). Puncturing assembly (700) of this example comprises a shaft (702) having a closed distal end (704) and a lateral aperture (706) formed proximal to closed distal end (704). A needle (710) is slidably disposed in a passageway extending longitudinally through shaft (702). Needle (710) has a sharp distal tip (712) and is laterally flexible, though needle (710) has enough compressive strength to be driven through inner shaft member (56) as will be described in greater detail below. Shaft (702) is sized to fit in inner lumen (57) of inner shaft member (56), and is slidable therein.

Puncturing assembly (700) is inserted within inner shaft member (56) with needle (710) in the retracted state, as shown in FIG. 14A, with lateral aperture (706) located at a longitudinal position associated with the interior of balloon (18, 58). With needle (710) in the retracted state, sharp distal tip (712) of needle (710) is entirely contained within shaft (702). As shown in FIG. 14B, needle (710) is advanced distally to drive sharp distal tip (712) into the sidewall of inner shaft member (56). In particular, as needle (710) is advanced distally, a ramp (708) within shaft (702) directs sharp distal tip (712) laterally and out through lateral aperture (706). Sharp distal tip (712) pierces inner shaft member (56) as needle (710) continues to advance distally after sharp distal tip (712) exits lateral aperture (706). In some versions, sharp distal tip (712) further pierces the outer wall of balloon (18, 58). Needle (710) is then translated proximally to draw sharp distal tip (712) back into shaft (702) as shown in FIG. 14C, leaving opening (55) in inner shaft member (56). Fluid may then drain from balloon (18, 58), through opening (55), and through inner shaft member (56). Puncturing assembly (700) may be removed from balloon catheter (10, 50) to more quickly drain balloon (18, 58), or puncturing assembly (700) may remain within balloon catheter (10, 50) while balloon (18, 58) drains.

H. Exemplary Heated Element

While the foregoing examples use sharp elements to pierce or puncture inner shaft (56) and/or balloon (18, 58), it should be understood that inner shaft (56) and/or balloon (18, 58) may be ruptured in numerous other ways. By way of example only, inner shaft (56) and/or balloon (18, 58) may be ruptured by overinflating balloon (18, 58), by applying a compressive force on the exterior of balloon (e.g., using a distally advancing sheath), by engaging inner shaft (56) and/or balloon (18, 58) with an ultrasonically vibrating element, and/or by engaging inner shaft (56) and/or balloon (18, 58) with a heating element.

FIGS. 15A-15B depict an exemplary rupturing assembly (800) that includes a heating element (802), which may be used to melt or otherwise burn an opening into inner shaft (56), thereby creating a path for fluid to drain from balloon (18, 58). Heating element (802) is secured to the interior of inner shaft (56) in this example and is in communication with a power source (806) via a wire (84). By way of example only, heating element (802) may be integrally formed in the sidewall of inner shaft (56), may be applied to the interior surface of inner shaft (56), may be applied to the exterior surface of inner shaft (56), or may otherwise be associated with inner shaft (56). It should be understood that heating element (802) is located at a longitudinal position associated with the interior of balloon (18, 58).

When it becomes desirable to deflate balloon (18, 58), power source (806) may be activated to cause heating element (802) to heat up. This may eventually melt or otherwise burn an opening (55) in inner shaft (56), as shown in FIG. 15B. Fluid may then drain from balloon (18, 58), through opening (55), and through inner shaft member (56). In some instances, the portion of inner shaft (56) providing structural support to heating element (802) melts away, such that heating element (802) disengages inner shaft (56). In such instances, heating element (802) may still receive structural support from wire (804). It should also be understood that heating element (802) and wire (804) may have sufficient electrical insulation to not have any electrical interaction with saline draining from balloon (18, 58) through opening (55). In addition or in the alternative, rupturing assembly (800) may include a fuse or other type of kill switch that automatically turns off power as soon as opening is formed (55) or as soon as heating element (802) comes in contact with saline.

While heating element (802) is described above as an integral feature of shaft member (56), it should be understood that heating element (802) may instead be incorporated into a stylet (22) or other elongate member that is selectively inserted through lumen (57) of inner shaft (56). It should also be understood that, regardless of whether heating element (802) is integrated into inner shaft (56) or provided on a member that is insertable into inner shaft (56), a plurality of heating elements (802) may be provided and activated simultaneously to create more than one opening (55) in inner shaft (56).

III. Exemplary External Puncturing Assembly

The examples described above relate to puncturing assemblies that are inserted through lumen (57) of inner shaft (56). Thus, as noted above, such puncturing assemblies may be incorporated into a stylet (22) or may be inserted into lumen (57) after stylet (22) has been removed from lumen (57). In some other variations of internal puncturing assemblies such as those described above, a separate dedicated lumen is provided within inner shaft (56), such that stylet (22) may be inserted into one lumen of inner shaft (56) while the puncturing assembly is inserted into another lumen of inner shaft (56). As another merely illustrative variation, an internal puncturing assembly may be inserted through a space defined between the interior of outer shaft (54) and the exterior of inner shaft (52). For instance, a dedicated lumen may be defined in this space for receipt of the internal puncturing assembly. Various other suitable ways in which an internal puncturing assembly may be positioned in and/or otherwise incorporated into dilation system (8) will be apparent to those of ordinary skill in the art in view of the teachings herein.

It should also be understood that dilation system (8) may incorporate an external puncturing assembly, in addition to or in lieu of having an internal puncturing assembly. Such an external puncturing assembly may include features positioned outside of outer shaft (54). Various examples of external puncturing assemblies will be described in greater detail below, while other examples of external puncturing assemblies will be apparent to those of ordinary skill in the art in view of the teachings herein.

A. Exemplary Laterally Placed External Puncturing Needle

FIGS. 16A-16C show an exemplary puncturing assembly (900) disposed on the exterior of outer shaft (54). Puncturing assembly (900) of this example comprises a sheath (902) secured to the exterior of outer shaft (54), with a needle (906) that is slidably received in sheath (902). As dilation system (8) is advanced through a patient's airway to position balloon (18, 58) at a stenosis site, needle (906) is in a retracted position such that the sharp distal tip (908) of needle (906) is located proximal to the open distal end (904) of sheath (902), as shown in FIG. 16A. When the operator wishes to rupture balloon (18, 58), the operator advances needle (906) distally, piercing balloon (18, 58) with sharp distal tip (908) as shown in FIG. 16B. The operator then retracts needle (906) proximally, leaving an opening (59) in balloon (18, 58) as shown in FIG. 16C. Fluid may then drain from balloon (18, 58), through opening (55). The draining fluid may simply drain through the patient's airway. Alternatively, the draining fluid may be pulled away using suction. For instance, suction may be provided through sheath (902) and/or through another suction tube.

It should also be understood that needle (906) is just one example of an instrument that may be inserted through sheath (902). By way of example only, numerous versions of the internal puncturing assemblies described above may be inserted through sheath (902) to selectively rupture balloon (18, 58). Other suitable instruments that may be inserted through sheath (902) will be apparent to those of ordinary skill in the art in view of the teachings herein.

B. Exemplary Coaxial External Cutting Sheath

FIGS. 17A-17C show an exemplary puncturing assembly (1000) disposed on the exterior of outer shaft (54). Puncturing assembly (1000) of this example comprises a sheath (1002) that is slidably disposed about the exterior of outer shaft (54). Sheath (1002) includes a rigid proximal portion (1004) and a deformable distal portion (1006). A plurality of sharp blades (1008) are secured to rigid proximal portion (1004) and extend distally from rigid proximal portion (1004), within deformable distal portion (1006). In some versions, blades (1008) are simply covered by deformable distal portion (1006). In some other versions, blades (1008) are embedded in deformable distal portion (1006).

As dilation system (8) is advanced through a patient's airway to position balloon (18, 58) at a stenosis site, sheath (1002) is in a proximal position and blades (1008) are covered or otherwise shielded by deformable distal portion (1006), as shown in FIG. 17A. Deformable distal portion (1006) thus prevents blades (1008) from snagging on the interior of the trachea or other anatomical structures during positioning of dilation system (8). When the operator wishes to rupture balloon (18, 58), the operator advances sheath (1002) distally, pressing deformable distal portion (1006) into the proximal end of balloon (18, 58). This causes deformable distal portion (1006) to deform, which causes blades (1008) to be driven into balloon (18, 58) as sheath (1002) continues to advance distally, as shown in FIG. 17B. Blades (1008) thus pierce balloon (18, 58). Fluid may then drain from balloon (18, 58), through openings (55). The draining fluid may simply drain through the patient's airway. Alternatively, the draining fluid may be pulled away using suction. For instance, suction may be provided through sheath (1002) and/or through another suction tube.

It should be understood that the material forming deformable distal portion (1006) deforms in response to less force than the force that is required to deform inflated balloon (18, 58). Various suitable materials that may used to form deformable distal portion (1006) will be apparent to those of ordinary skill in the art in view of the teachings herein. It should also be understood that deformable distal portion (1006) may be replaced with a retractable sheath; or that blades (1008) may be selectively retractable relative to rigid proximal portion (1004). Furthermore, while a series of separate blades (1008) are shown, it should be understood that a single blade may instead be used. Still other suitable variations will be apparent to those of ordinary skill in the art in view of the teachings herein.

IV. Miscellaneous

It should be appreciated that any patent, publication, or other disclosure material, in whole or in part, that is said to be incorporated by reference herein is incorporated herein only to the extent that the incorporated material does not conflict with existing definitions, statements, or other disclosure material set forth in this disclosure. As such, and to the extent necessary, the disclosure as explicitly set forth herein supersedes any conflicting material incorporated herein by reference. Any material, or portion thereof, that is said to be incorporated by reference herein, but which conflicts with existing definitions, statements, or other disclosure material set forth herein will only be incorporated to the extent that no conflict arises between that incorporated material and the existing disclosure material.

Versions of the devices disclosed herein can be designed to be disposed of after a single use, or they can be designed to be used multiple times. Versions may, in either or both cases, be reconditioned for reuse after at least one use. Reconditioning may include any combination of the steps of disassembly of the device, followed by cleaning or replacement of particular pieces, and subsequent reassembly. In particular, versions of the device may be disassembled, and any number of the particular pieces or parts of the device may be selectively replaced or removed in any combination. Upon cleaning and/or replacement of particular parts, versions of the device may be reassembled for subsequent use either at a reconditioning facility, or by a surgical team immediately prior to a surgical procedure. Those skilled in the art will appreciate that reconditioning of a device may utilize a variety of techniques for disassembly, cleaning/replacement, and reassembly. Use of such techniques, and the resulting reconditioned device, are all within the scope of the present application.

By way of example only, versions described herein may be processed before surgery. First, a new or used instrument may be obtained and if necessary cleaned. The instrument may then be sterilized. In one sterilization technique, the instrument is placed in a closed and sealed container, such as a plastic or TYVEK bag. The container and instrument may then be placed in a field of radiation that can penetrate the container, such as gamma radiation, x-rays, or high-energy electrons. The radiation may kill bacteria on the instrument and in the container. The sterilized instrument may then be stored in the sterile container. The sealed container may keep the instrument sterile until it is opened in a surgical facility. A device may also be sterilized using any other technique known in the art, including but not limited to beta or gamma radiation, ethylene oxide, or steam.

Having shown and described various versions of the present invention, further adaptations of the methods and systems described herein may be accomplished by appropriate modifications by one of ordinary skill in the art without departing from the scope of the present invention. Several of such potential modifications have been mentioned, and others will be apparent to those skilled in the art. For instance, the examples, versions, geometrics, materials, dimensions, ratios, steps, and the like discussed above are illustrative and are not required. Accordingly, the scope of the present invention should be considered in terms of the following claims and is understood not to be limited to the details of structure and operation shown and described in the specification and drawings.

I/we claim:

1. A dilation system, wherein the dilation system comprises:
(a) a dilation catheter, wherein the dilation catheter comprises:
(i) a shaft,
(ii) a dilator coupled to a distal end of the shaft, wherein the dilator is configured dilate from a collapsed state to an expanded state, and
(iii) a lumen extending through the shaft and the dilator; and
(b) a puncturing assembly, wherein the puncturing assembly comprises at least one puncturing member, wherein the puncturing assembly is configured to be positioned within the lumen of the dilation catheter, wherein the puncturing assembly is configured to transition from a first position to a second position, wherein the puncturing assembly is configured to translate within the lumen in the first position, wherein the puncturing assembly is configured to puncture the lumen in the second position.

2. The dilation system of claim 1, wherein the puncturing assembly is configured to return from the second position to the first position in response to puncturing of the lumen.

3. The dilation system of claim 1, wherein the dilation system is configured to be positioned in an anatomical airway.

4. The dilation system of claim 1, wherein the puncturing assembly is configured to further puncture the dilator in the second position.

5. The dilation system of claim 1, wherein the puncturing assembly comprises an expandable tube, wherein the at least one puncturing member is positioned on a distal end of the tube.

6. The dilation system of claim 5, wherein the puncturing assembly comprises a mandrel having a tapered configuration, wherein the mandrel is translatable to expand the tube.

7. The dilation system of claim 1, wherein the puncturing assembly comprises an expandable tube, wherein the puncturing assembly comprises a shaft, wherein the shaft is positioned within the tube, wherein the at least one puncturing member is positioned on a distal end of the shaft.

8. The dilation system of claim 7, wherein the at least one puncturing member is pivotable relative to the shaft, wherein the at least one puncturing member is configured to pivot outwardly from the shaft in response to movement of the shaft relative to the tube.

9. The dilation system of claim 1, wherein the puncturing assembly comprises an expandable tube, wherein the at least one puncturing member is configured to be resiliently biased outwardly from the tube, wherein the at least one puncturing member is configured to be positioned within the tube.

10. The dilation system of claim 9, wherein the at least one puncturing member is configured to bias beyond the tube in response to the at least one puncturing member being exposed from the tube.

11. The dilation system of claim 1, wherein the puncturing assembly comprises an actuator, wherein the at least one puncturing member is configured to expand from the first position to the second position in response to movement of the actuator.

12. The dilation system of claim 11, wherein the actuator is configured to rotate, wherein the at least one puncturing member is configured to rotate in response to rotation of the actuator.

13. The dilation system of claim 11, wherein the puncturing assembly comprises a shaft having a tapered portion, wherein the shaft is configured to translate in response to translation of the actuator, wherein the tapered portion of the shaft is configured to expand the at least one puncturing member from the first position to the second position.

14. The dilation system of claim 11, wherein the puncturing assembly comprises a blade, wherein the blade is configured to pivot from the first position to the second position in response to movement of the actuator.

15. The dilation system of claim 1, wherein the puncturing assembly comprises a heating element operable to melt a portion of the shaft to form a side opening in the lumen.

16. The dilation system of claim 1, wherein the dilator comprises a balloon.

17. A puncturing assembly, wherein the puncturing assembly comprises:
(a) a dilation catheter, wherein the dilation catheter comprises:
(i) a shaft, wherein the shaft defines a longitudinal axis, and
(ii) a dilator coupled to a distal end of the shaft, wherein the dilator is configured dilate from a collapsed state to an expanded state,
(b) a puncturing assembly, wherein the puncturing assembly is located external to the shaft, wherein the puncturing assembly operable to puncture the dilator, wherein the puncturing assembly is oriented parallel to the longitudinal axis of the shaft.

18. The puncturing assembly of claim 17, wherein the puncturing assembly comprises:
(i) a sheath secured to the exterior of the shaft, and
(ii) a translating member slidably disposed in the sheath, wherein the translating member is translatable relative to the sheath to puncture the dilator.

19. The puncturing assembly of claim 17, wherein the puncturing assembly comprises:
(i) a sheath aligned along the longitudinal axis of the shaft, and
(ii) a sharp puncturing member secured to the sheath, wherein the sheath is translatable relative to the shaft to drive the sharp puncturing member into the dilator.

20. A puncturing assembly, wherein the puncturing assembly comprises:
(a) a dilation assembly, wherein the dilation assembly comprises:
(i) a shaft, wherein the shaft defines a lumen, wherein the shaft further defines a longitudinal axis,
(ii) a dilator coupled to a distal end of the shaft, wherein the lumen extends through an interior region of the dilator;
(b) a tube, wherein the tube is parallel with the longitudinal axis of the shaft; and
(c) at least one puncturing member, wherein the at least one puncturing member is configured to be positioned within the tube, wherein the at least one puncturing member is movable from a first position to a second position, wherein the at least one puncturing member is configured to create an opening to drain fluid from the dilator upon movement of the at least one puncturing member from the first position to the second position.
